(19) United States
(12) Patent Application Publication
(10) Pub. No.: US 2009/0093068 A1
(43) Pub. Date: Apr. 9, 2009

(54) IMMUNOSENSOR AND MEASURING METHOD USING THE SAME

(75) Inventors: Keiko Yugawa, Nara (JP); Shinji Tanaka, Osaka (JP); Shin Ikeda, Osaka (JP)

Correspondence Address: MCDERMOTT WILL & EMERY LLP, 600 13TH STREET, NW, WASHINGTON, DC, 20005-3096, US

(73) Assignee: PANASONIC CORPORATION, Osaka (JP)

(21) Appl. No.: 12/330,859

(22) Filed: Dec. 9, 2008

Related U.S. Application Data
(63) Continuation of application No. PCT/JP2007/068444, filed on Sep. 21, 2007.

(30) Foreign Application Priority Data
Sep. 26, 2006 (JP) 2006-260168
Sep. 26, 2006 (JP) 2006 260169

(51) Int. Cl. G01N 33/53; B01J 19/00

(52) U.S. Cl. 436/501; 422/68.1

(57) ABSTRACT

An immunosensor includes a base body (101) having a sample holding portion (102) which holds a test sample, a sample introducing port (103) which is communicated with the sample holding portion (102) and through which the test sample is introduced to the sample holding portion (102), a dried first reagent body (109) which contains an antibody to a material to be measured which is contained in the test sample, and a dried second reagent body (110) which contains polyethylene glycol, and in the sample holding portion (102), the first reagent body (109) is placed closer to the sample introducing port (103) than the second reagent body (110).

### TECHNICAL FIELD

The present invention relates to an immunosensor and a measuring method using the same, and particularly to the configuration of the immunosensor.

### BACKGROUND ART

Conventionally known as methods for easily measuring a component in a sample are turbidimetric immunoassay and nephelometric immunoassay, each of which optically measures an aggregate generated by an antigen-antibody reaction using an antibody which specifically recognizes the component (protein) in the sample. Moreover, known is an immune reaction measuring reagent kit which can easily improve a measured value and obtain high measurement sensitivity when measuring the component in the sample by the turbidimetric immunoassay or the nephelometric immunoassay (see Patent Document 1 for example).

The immune reaction measuring reagent kit disclosed in Patent Document 1 is configured such that in the case of forming a reaction system containing the sample containing a material to be measured as a component to be measured, the antibody to the material to be measured, and phthalic acid or a salt of phthalic acid, the pH of the reaction system is set to be less than 7. With this, the measured value can be easily improved and high measurement sensitivity can be obtained without increasing the viscosity of the reaction system (solution). Note that Patent Document 1 discloses that polyethylene glycol (hereinafter referred to as “PEG”) is added to the reaction system in order to accelerate the antigen-antibody reaction and highly sensitively measure a minor component.

Moreover, known as a sensor which measures the component in the sample by the turbidimetric immunoassay or the nephelometric immunoassay is a sensor in which a dried antibody reagent is placed inside a container constituting the sensor (see Patent Documents 2 and 3 for example). The sensor disclosed in Patent Document 2 includes a sample holding portion which holds the sample, a sample introducing port through which the sample is supplied to the sample holding portion, and a reagent holding portion which is formed inside the sample holding portion. Specifically, the reagent holding portion is formed by attaching a glass fiber carrier, which supports a dried anti-human albumin antibody, to an inner peripheral surface of the container constituting the sample holding portion. Note that Patent Document 2 discloses in Example that the measurement was carried out using the nephelometric immunoassay by adding polyethylene glycol to an antigen-antibody reaction system in order to examine how the addition of NaCL, KCL, and CaCL₃ affects the reaction system.

Patent Document 3 discloses a blood test container including a tubular container, a second tubular container which is smaller in diameter than the tubular container, a blood test measuring reagent which is fixed to a gap between the tubular container and the second tubular container, and a seal material which seals the gap. In the blood test container, the seal material is fixed at a position which is located on an upper side of the blood test measuring reagent and between the vicinity of an upper end of an outer peripheral surface of the second tubular container and an inner peripheral surface of the tubular container Patent Document 3 further discloses that a freeze-dried antibody is used as the blood test measuring reagent, and polyethylene glycol is used as the seal material.

Patent Document 1: Pamphlet of International Publication No. 03/056333

Patent Document 2: Pamphlet of International Publication No. 2005/108960

Patent Document 3: Japanese Laid-Open Patent Application Publication No. 2000-074910

### DISCLOSURE OF THE INVENTION

### Problems to be Solved by the Invention

However, how to add the PEG is not disclosed in Patent Document 1 which discloses the immune reaction measuring reagent kit or in Patent Document 2 which discloses the sensor. Moreover, the blood test container disclosed in Patent Document 3 has such a problem that since the seal material, i.e., the PEG is first dissolved in the sample, the sample increases in viscosity, so that the reagent, i.e., the antibody is less likely to be dissolved in the sample.

The present invention was made to solve the above problems, and an object of the present invention is to provide an immunosensor capable of measuring the concentration of a material to be measured, which is contained in a test sample, and a measuring method using this immunosensor.

### Means for Solving the Problems

The present inventors have found that in a case where a dried antibody and dried PEG are supported in a mixed state in the immunosensor, a sensor response which depends on the concentration of an antigen contained in the test sample cannot be obtained. Then, the present inventors have found that defining the positional relation between the antibody and the PEG is highly effective to achieve the above object of the present invention. Thus, the present invention was achieved.

To be specific, an immunosensor according to the present invention includes: a container-like base body whose internal space forms a sample holding portion which holds a test sample; a sample introducing port which is formed on the base body to be communicated with the sample holding portion; a dried first reagent body which contains an antibody to a material to be measured which is contained in the test sample; and a dried second reagent body which contains polyethylene glycol, wherein in the sample holding portion, the first reagent body is placed closer to the sample introducing port than the second reagent body.

With this, since the first reagent body contacts the test sample before the second reagent body containing polyethylene glycol, the antibody contained in the first reagent body can be easily dissolved in the test sample. Moreover, since the antibody is easily dissolved, it adequately reacts with the material to be measured (antigen) which is contained in the test sample. Therefore, the concentration of the material to be measured which is contained in the test sample can be accurately measured.

Moreover, in the immunosensor according to the present invention, the first reagent body may be placed to be adhered to an inner surface of the base body.

Moreover, in the immunosensor according to the present invention, a portion of the second reagent body which is opposed to the first reagent body may have a portion which projects toward the second reagent body.

Moreover, in the immunosensor according to the present invention, a portion of the second reagent body which is opposed to the first reagent body may have a spherical shape.

Moreover, in the immunosensor according to the present invention, it is preferable that the second reagent body contain a metal salt of phthalic acid.

Moreover, in the immunosensor according to the present invention, the metal salt of phthalic acid may be potassium hydrogen phthalate.

Moreover, in the immunosensor according to the present invention, a weight ratio of the potassium hydrogen phthalate to the polyethylene glycol may be not less than 0.26 and not more than 1.02.

Moreover, in the immunosensor according to the present invention, the second reagent body may contain a salt selected from the group consisting of trisodium citrate, disodium succinate, sodium chloride, and potassium chloride.

Further, in the immunosensor according to the present invention, the base body may have a light transmitting portion which transmits light such that the light penetrates a wall forming the base body.

Moreover, a measuring method using an immunosensor according to the present invention is a measuring method using an immunosensor including: a container-like base body whose internal space forms a sample holding portion which holds a test sample; a sample introducing port which is formed on the base body to be communicated with the sample holding portion; a dried first reagent body which contains an antibody to a material to be measured which is contained in the test sample; and a dried second reagent body which contains polyethylene glycol, wherein in the sample holding portion, the first reagent body is placed closer to the sample introducing port than the second reagent body, and the method includes the step of introducing the test sample through the sample introducing port to the sample holding portion, wherein: the test sample introduced into the sample holding portion contacts the first reagent body; the first reagent body is dissolved in the test sample; the test sample in which the first reagent body is dissolved contacts the second reagent body; and the second reagent body is dissolved in the test sample.

With this, since the first reagent body contacts the test sample before the second reagent body containing polyethylene glycol, the antibody contained in the first reagent body can be easily dissolved in the test sample. Moreover, since the antibody is easily dissolved, it adequately reacts with the material to be measured (antigen) which is contained in the test sample. Therefore, the concentration of the material to be measured which is contained in the test sample can be accurately measured.

Moreover, in the measuring method using the immunosensor according to the present invention, a concentration of the polyethylene glycol in an entire amount of the test sample introduced into the sample holding portion may be not less than 1 weight % and not more than 15 weight %.

### EFFECTS OF THE INVENTION

In accordance with the immunosensor according to the present invention and the measuring method using this immunosensor, it is possible to, with a simple configuration, suppress the deterioration of the antibody and accurately measure the concentration of the material to be measured, which is contained in the test sample.

### BRIEF DESCRIPTION OF THE DRAWINGS

FIG. 1 is a perspective view schematically showing the configuration of an immunosensor according to Embodiment 1 of the present invention.

FIG. 2 is a cross-sectional view schematically showing a cross section taken along line II-II of the immunosensor shown in FIG. 1.

FIG. 3 is a perspective view schematically showing the configuration of a measuring device which uses the immunosensor according to Embodiment 1 of the present invention.

FIG. 4 is a block diagram schematically showing a functional configuration of the measuring device shown in FIG. 3.

FIG. 5 is a flow chart schematically showing a method for measuring a material to be measured by the measuring device which uses the immunosensor according to Embodiment 1 of the present invention.

FIG. 6 shows results of Evaluation Test 1 regarding the immunosensor of Example 1 and the immunosensor of Comparative Example 1.

FIG. 7 shows measurement results, which are obtained by ELISA, regarding the immunosensor of Example 2 and the immunosensor of Comparative Example 3.

FIG. 8 shows measurement results of a dissolution rate of PEG in an immunosensor 100 of Example 3.

FIG. 9 shows results of a solubility test in Evaluation Test 5.

### EXPLANATION OF REFERENCE NUMBERS

100 immunosensor
101 base body
102 space (sample holding portion)
103 through hole (sample introducing port)
104 suction port
105 first surface
106 second surface (light incident portion)
107 third surface (light emanating portion)
108 fourth surface
109 first reagent body
110 second reagent body
111 light transmitting portion
300 measuring device
301 immunosensor attaching portion
302 display portion
303 sample suction start button
304 immunosensor detach button
305 sensor attaching port
401 controller
404 piston mechanism
406 timer portion
407 light source
408 photoreceiver
409 memory
410 immunosensor detach mechanism
411 recording portion
412 sending portion
413 receiving portion

### BEST MODE FOR CARRYING OUT THE INVENTION

Hereinafter, the best mode for carrying out the present invention will be explained in reference to the drawings. In the drawings, same reference numbers are used for the same or corresponding members, and a repetition of the same explanation is avoided.

### Embodiment 1

Embodiment 1 of the present invention exemplifies a case where a test sample is urine, a material to be measured is human albumin, and the material to be measured is detected by turbidimetric immunoassay.

Configuration of Immunosensor

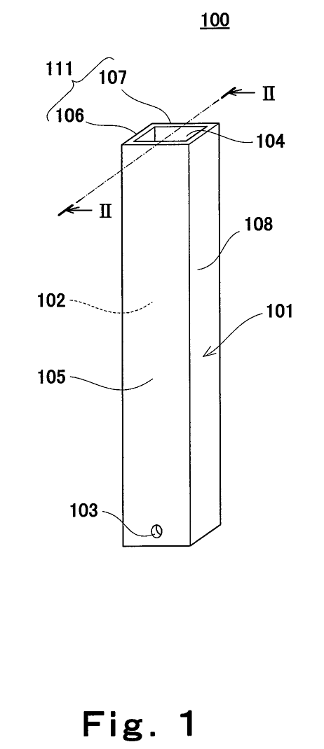

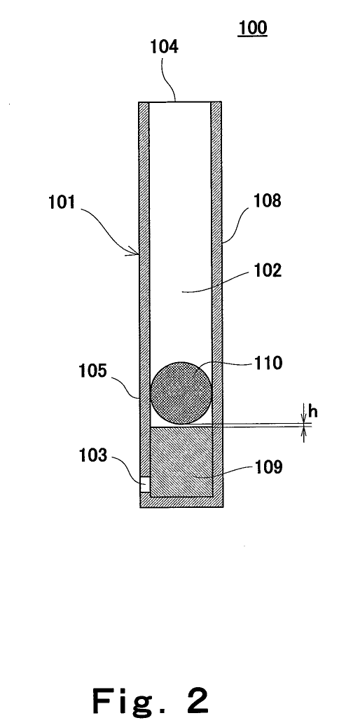

First, the configuration of the immunosensor according to Embodiment 1 of the present invention will be explained in reference to FIGS. 1 and 2.

FIG. 1 is a perspective view schematically showing the configuration of the immunosensor according to Embodiment 1 of the present invention. FIG. 2 is a cross-sectional view schematically showing a cross section taken along line II-II of the immunosensor shown in FIG. 1.

As shown in FIGS. 1 and 2, an immunosensor 100 according to Embodiment 1 of the present invention includes a base body 101 which is transparent and made of polystyrene. The base body 101 is formed by a rectangular solid container, and has first to fourth surfaces 105 to 108. Inside the base body 101, a space 102 (hereinafter referred to as a sample holding portion 102) which holds the test sample is formed. The base body 101 is closed at one end portion thereof, and opens to outside at the other end portion thereof. The open end portion serves as a suction port 104.

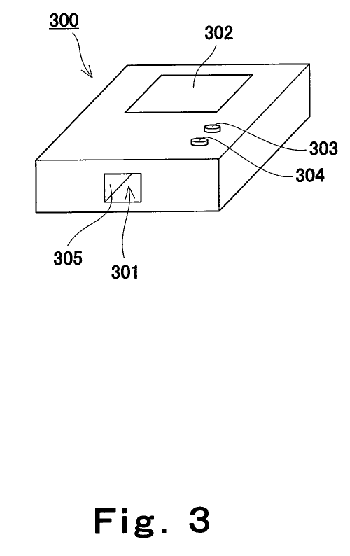

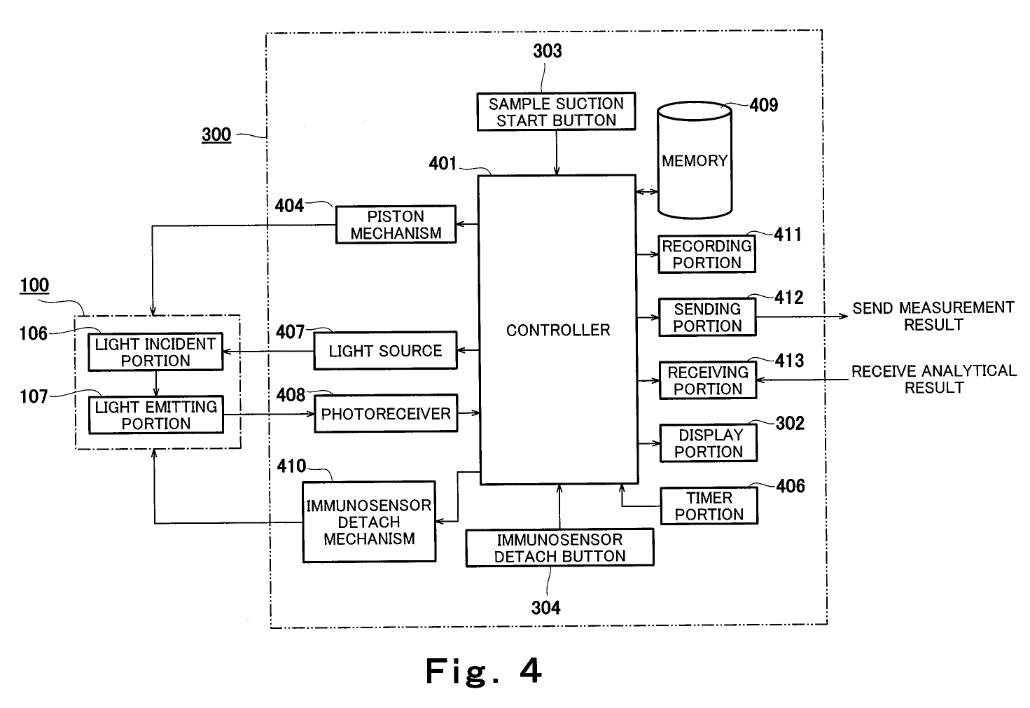

Moreover, a through hole 103 which penetrates the first surface 105 of the base body 101 in a thickness direction is formed at a lower portion of the first surface 105 of the base body 101, and serves as a sample introducing port 103. In the embodiment of the present invention, as will be described later, the immunosensor 100 is attached to a measuring device 300, a part of the immunosensor 100 is, for example, immersed in the urine stored in a base body, and then, air inside the sample holding portion 102 is suctioned through the suction port 104 by a piston mechanism 404 (see FIGS. 3 and 4) of the measuring device 300. With this, the urine as the test sample can be introduced into the sample holding portion 102.

Moreover, at a lower portion of the sample holding portion 102 of the base body 101, a rectangular solid first reagent body 109 containing an antibody (herein, an anti-human albumin antibody) is placed, and a spherical second reagent body 110 containing polyethylene glycol (hereinafter referred to as PEG) is placed above the first reagent body 109. In other words, the first reagent body 109 is placed separately from the second reagent body 110 and closer to the sample introducing port 103 than the second reagent body 110. With this, since the test sample introduced from the sample introducing port 103 dissolves the first reagent body 109 before the second reagent body 110, the viscosity of the test sample is not increased by the PEG contained in the second reagent body 110, so that the test sample can easily dissolve the antibody contained in the first reagent body.

As defined herein, the first reagent body 109 is produced by freeze-drying a solution containing an antibody, and the second reagent body 110 is produced by freeze-drying a solution containing the PEG. Moreover, the phrase that the first reagent body 109 and the second reagent body 110 are placed separately from each other means that the antibody and the PEG are contained in the first reagent body 109 and the second reagent body 110, respectively, as single compounds, and that a freeze-dried mixture of the antibody and the PEG is not contained in them.

Moreover, among four surfaces constituting the outer peripheral surface of the base body 101, the second surface 106 serves as a light incident portion 106, and the third surface 107 serves as a light emanating portion 107. In the embodiment of the present invention, the second surface (light incident portion) 106 and the third surface (light emanating portion) 107 constitute a light transmitting portion 111 which optically measures the test sample held by the sample holding portion 102.

It is preferable that the light incident portion 106 and the light emanating portion 107 be formed by an optically transparent material or a material which does not substantially absorb visible light. Examples of such materials are quartz, glass, polystyrene, and polymethylmethacrylate. Especially in a case where the immunosensor 100 is formed as a disposable type, it is preferable that the immunosensor 100 be formed by polystyrene from the standpoint of cost.

The embodiment of the present invention is configured such that the base body 101 is entirely transparent. However, the embodiment of the present invention is not limited to this. The embodiment of the present invention may be configured such that a portion (light incident portion 106) which is subjected to light emitted from a light source 407 of the measuring device 300 described below and a portion (light emanating portion 107) which emanates the light from the base body 101 to a photoreceiver 408 of the measuring device 300 are transparent. Since the embodiment of the present invention uses the turbidimetric immunoassay which detects scattered light of the light incident on the light incident portion 106 of the base body 101, the light emanating portion 107 is formed so as not to be opposed to the light incident portion 106. However, in the case of detecting the material to be measured by the nephelometric immunoassay, the light incident portion 106 and the light emanating portion 107 are formed to be opposed to each other.

Moreover, in the embodiment of the present invention, it is preferable that the immunosensor 100 be detachably attached to an immunosensor attaching portion 301 of the measuring device 300 described below. Moreover, it is preferable that the immunosensor 100 be disposable in order to realize accurate measurement of the material to be measured, which is contained in the test sample.

Configurations of First Reagent Body and Second Reagent Body

Next, the configurations of the first reagent body 109 and the second reagent body 110 in the immunosensor 100 according to Embodiment 1 of the present invention will be explained in detail in reference to FIG. 2.

In order to more easily dissolve the antibody contained in the first reagent body 109, it is preferable that the first reagent body 109 be placed to be adhered to an inner peripheral surface of the base body 101, and it is more preferable that the first reagent body 109 be adhered to the inner peripheral surface and a bottom surface of the base body 101. Moreover, in order to suppress the deterioration of the antibody contained in the first reagent body 109, it is preferable that the first reagent body 109 and the second reagent body 110 be placed separately from each other. Further, in order to more easily dissolve the antibody contained in the first reagent body 109, it is preferable that the first reagent body 109 and the second reagent body 110 be placed close to each other.

Therefore, in order to suppress the deterioration of the antibody contained in the first reagent body 109 and more easily dissolve the antibody, it is preferable that a distance h between the first reagent body 109 and the second reagent body 110 be short. Note that the first reagent body 109 and the second reagent body 110 may be placed to contact each other. In this case, in order to reduce a contact area between the first reagent body 109 and the second reagent body 110, it is preferable that a portion of the second reagent body 110 which is opposed to an upper surface of the first reagent body 109 project downward, and it is more preferable that the portion of the second reagent body 110 be formed in a spherical shape.

The antibody contained in the first reagent body 109 may be a polyclonal antibody or a monoclonal antibody. Moreover, the antibody contained in the first reagent body 109 may be a combination of a plurality of monoclonal antibodies. The polyclonal antibody is easily produced. In contrast, quality control of the monoclonal antibody is easy since the same antibody can be obtained by producing an antibody producing cell. Moreover, examples of the antibody contained in the first reagent body 109 are an antibody to protein, such as albumin and C reactive protein (CRP), contained in the urine, and an antibody to hormone, such as human chorionic gonadotropin (hCG: human pregnancy hormone) and LH (luteinizing hormone), contained in the urine. In the case of using the anti-human albumin antibody, in order to properly cause the antigen-antibody reaction with the albumin in the sample, it is preferable that the anti-human albumin antibody be contained in the first reagent body 109 such that the amount thereof is 0.1 to 20 mg/mL with respect to the entire amount of the test sample introduced to the reagent holding portion 102 of the base body 100.

It is preferable that a polymerization degree of the PEG contained in the second reagent body 110 be 158 to 204 and an average molecular weight of the PEG contained in the second reagent body 110 be 7,000 to 9,000, since the PEG contained in the second reagent body 110 is less likely to cause nonspecific agglutination with a material other than the material to be measured. Moreover, it is preferable that the amount of the PEG contained in the second reagent body 110 be 1 weight % or more with respect to the entire amount of the test sample introduced into the reagent holding portion 102 of the base body 100, since a satisfactory effect of promoting the agglutination can be obtained by the PEG. In order to realize an appropriate viscosity of the sample, it is preferable that the amount of the PEG contained in the second reagent body 110 be 15 weight % or less. From these standpoints, it is more preferable that the amount of the PEG contained in the second reagent body 110 be 4 weight %.

Further, in order to amplify a response value of the turbidimetric immunoassay, to be specific, in order to easily improve the measured value and obtain high measurement sensitivity when carrying out the measurement by the measuring device 300, it is preferable that the second reagent body 110 contain a metal salt of phthalic acid. Examples of the metal salt of phthalic acid are a potassium salt of phthalic acid and a sodium salt of phthalic acid, and these metal salts are preferable since these salts are easily dissolved in water. Moreover, it is more preferable that the second reagent body 110 contain potassium hydrogen phthalate, since it amplifies the response value of the turbidimetric immunoassay and has high solubility in water.

In order to increase the solubility of the PEG, a ratio Y/X of a weight Y of the potassium hydrogen phthalate to a weight X of the PEG contained in the second reagent body 110 is preferably 0.26 to 1.02, and more preferably 0.26 to 0.51.

Moreover, in order to increase the solubility of the PEG contained in the second reagent body 110, the second reagent body 110 may include one salt selected from the group consisting of trisodium citrate, disodium succinate, sodium chloride, and potassium chloride.

It is estimated that the addition of the salt to the PEG increases the solubility of the PEG because of reasons below. To be specific, it is estimated that by adding salt to the PEG and freeze drying it, a solid (hereinafter referred to as a salt-containing high polymer compound) having such a minute structure that the salt surrounds a poorly-soluble high polymer compound is formed. In the case of selecting a readily-soluble salt, when the test sample (aqueous solution) contacts the salt-containing high polymer compound, the salt immediately absorbs the water, so that the water surrounds the salt-containing high polymer compound. Therefore, it is estimated that the solubility of the high polymer compound (PEG) increases. Moreover, the high polymer compounds having the same polarity are prevented from agglutinating by the salt. Thus, it is estimated that the solubility of the PEG increases.

Method for Producing Immunosensor

Next, a method for producing the immunosensor 100 according to Embodiment 1 of the present invention will be explained.

First, a die having the shape of the base body 101 shown in FIGS. 1 and 2 is produced, and a liquid material (polystyrene for example) constituting the base body 101 is poured into the die. Thus, the base body 101 is produced. At this time, a transparent material may be dissolved and poured into the die such that the entire base body 101 is transparent, or the base body 101 may be produced such that only the transmitting portion 111 is transparent.

Next, an anti-albumin antibody reagent solution is prepared by adding an anti-albumin antibody reagent (8 mg/mL) to a 50 mM potassium hydrogen phthalate aqueous solution (pH 5.0). After the sample supplying port 103 of the base body 101 is closed by an adhesion tape, the anti-albumin antibody reagent solution is poured through the suction port 104 to the lower portion of the reagent holding portion 102, and the base body 101 is placed in a freezer at −80° C. With this, the anti-albumin antibody reagent solution freezes, and adheres to the inner peripheral surface and bottom surface of the base body 101. Thus, the first reagent body 109 is produced at the lower portion of the reagent holding portion 102.

Next, a 250 mM potassium hydrogen phthalate aqueous solution (pH 5.0) is stirred while adding thereto the PEG until the concentration of the PEG reaches 20 weight %. Thus, a PEG reagent solution is prepared. Next, the base body 101 in which the first reagent body 109 is produced is placed in a container which contains liquid nitrogen, and the PEG reagent solution is poured through the suction port 104. With this, without unfreezing the anti-albumin antibody contained in the first reagent body 109, the PEG reagent solution having a spherical shape is placed on the first reagent body 109 so as to contact the first reagent body 109. Since the PEG reagent solution immediately freezes, the second reagent body 110 is produced to contact the first reagent body 109.

Note that the second reagent body 110 may be placed in the reagent holding portion 102 in such a manner that the PEG reagent solution is dropped into the container which contains the liquid nitrogen, the PEG reagent solution is frozen to produce the second reagent body 110 having the spherical shape, and this second reagent body 110 is pressed into the reagent holding portion 102 through the suction port 104. By pressing the second reagent body 110 into the reagent holding portion 102, the first reagent body 109 and the second reagent body 110 can be placed separately from each other in the reagent holding portion 102.

Next, the base body 101 in which the first and second reagent bodies 109 and 110 are placed in the reagent holding portion 102 is immediately placed in a chamber of a freeze dryer, and is freeze-dried overnight. Thus, the immunosensor 100 is produced.

Configuration of Measuring Device

Next, the measuring device which uses the immunosensor 100 according to Embodiment 1 of the present invention will be explained in reference to FIGS. 3 and 4. Note that the measuring device itself according to the embodiment of the present invention is the same in configuration as a known measuring device, so that a detailed explanation of the configuration of the measuring device is omitted below.

FIG. 3 is a perspective view schematically showing the configuration of the measuring device which uses the immunosensor 100 according to Embodiment 1 of the present invention. FIG. 4 is a block diagram schematically showing a functional configuration of the measuring device shown in FIG. 3.

As shown in FIG. 3, the measuring device 300 which uses the immunosensor 100 according to Embodiment 1 of the present invention includes the immunosensor attaching portion 301, a display portion 302, a sample suction start button 303, and an immunosensor detach button 304. The immunosensor attaching portion 301 has a sensor attaching port 305 to which the suction port 104 of the immunosensor 100 is detachably attached. The piston mechanism 404 (see FIG. 4) including a cylinder (not shown) and a piston (not shown) which slides in the cylinder is disposed inside the sensor attaching port 305. Air is suctioned from the suction port 104 by the piston of the piston mechanism 404. Thus, the test sample is introduced into the reagent holding portion 102 of the immunosensor 100.

Moreover, the display portion 302 that is a display which shows measurement results, the sample suction start button 303, and the immunosensor detach button 304 are formed on a main surface of the measuring device 300.

As shown in FIG. 4, the light source 407, the photoreceiver 408, the piston mechanism 404, and an immunosensor detach mechanism 410 are formed inside the measuring device 300. The light source 407 is configured to emit light which is incident on the light incident portion 106 of the immunosensor 100 attached to the immunosensor attaching portion 301. The photoreceiver 408 is configured to receive light emanated from the light emanating portion 107 of the immunosensor 100.

The piston mechanism 404 is configured to cause the piston to move forward and backward by a linear stepping motor. The immunosensor detach mechanism 410 is configured to allow the immunosensor 100 to be detached from the measuring device 300 when an operator presses the immunosensor detach button 304. Although the piston mechanism 404 herein is configured to cause the piston to move forward and backward by the linear stepping motor, the present embodiment is not limited to this. The piston mechanism 404 may be configured to cause the piston to move forward and backward manually. Examples of the mechanism which causes the piston to move forward and backward manually are conventional syringes and conventional dispensers. The configuration of causing the piston to move forward and backward may be manual or automatic. However, the configuration of causing the piston move forward and backward automatically is preferable since the burden of the operator can be reduced. Moreover, the linear stepping motor does not have to be used as a power source which causes the piston to move forward and backward in the piston mechanism 404, and a general power source, such as a stepping motor or a direct-current motor, may be used.

Here, the stepping motor is a motor whose rotor rotates at a specific rotation angle in response to one pulse input signal, and can determine the rotation angle of the rotor in accordance with the number of input pulses. Therefore, the stepping motor does not require an encoder for positioning. To be specific, the stepping motor is a motor capable of suitably controlling a movement distance of the piston in accordance with the number of input pulses. Forward and backward movements of the piston by the stepping motor is realized by converting the rotational movement of the rotor of the stepping motor into a translatory movement by, for example, a gear mechanism and a translatory mechanism configured by combining a male screw and a female screw. In order to cause the piston to move forward and backward by using the direct-current motor, for example, the translatory mechanism which converts the rotational movement of the rotor into the translatory movement is required. Moreover, in order to appropriately control the movement distance of the piston by using the direct-current motor, the encoder which detects a rotational position of the rotor is required.

Meanwhile, the linear stepping motor incorporates the translatory mechanism configured by combining the male screw and the female screw, and is configured such that a rod-like movable portion thereof carries out the translatory movement in accordance with the number of input pulses. Therefore, the piston mechanism 404 can be configured by directly coupling the piston to the rod-like movable portion. On this account, the piston mechanism 404 can be comparatively simplified in configuration.

Further, the measuring device 300 includes: a controller 401 having a calculating portion which detects or quantitates the material to be measured, which is contained in the test sample, based on the light which is emanated from the light emanating portion 107 of the immunosensor 100 and received by the photoreceiver 408; a memory 409 which stores data regarding a calibration curve which shows a correlation between the concentration of the human albumin that is the material to be measured and the intensity of the light which is emanated from the light emanating portion 107 and received by the photoreceiver 408; a recording portion 411 which records the measurement results; a sending portion 412 which sends the measurement results to outside; a receiving portion 413 which receives an analytical result from outside; and a timer portion 406 which measures an elapsed time.

Measuring Method Using Immunosensor

Next, the method for measuring the material to be measured, which is contained in the test sample, by the measuring device 300 which uses the immunosensor 100 according to Embodiment 1 of the present invention will be explained in reference to FIGS. 1 to 5.

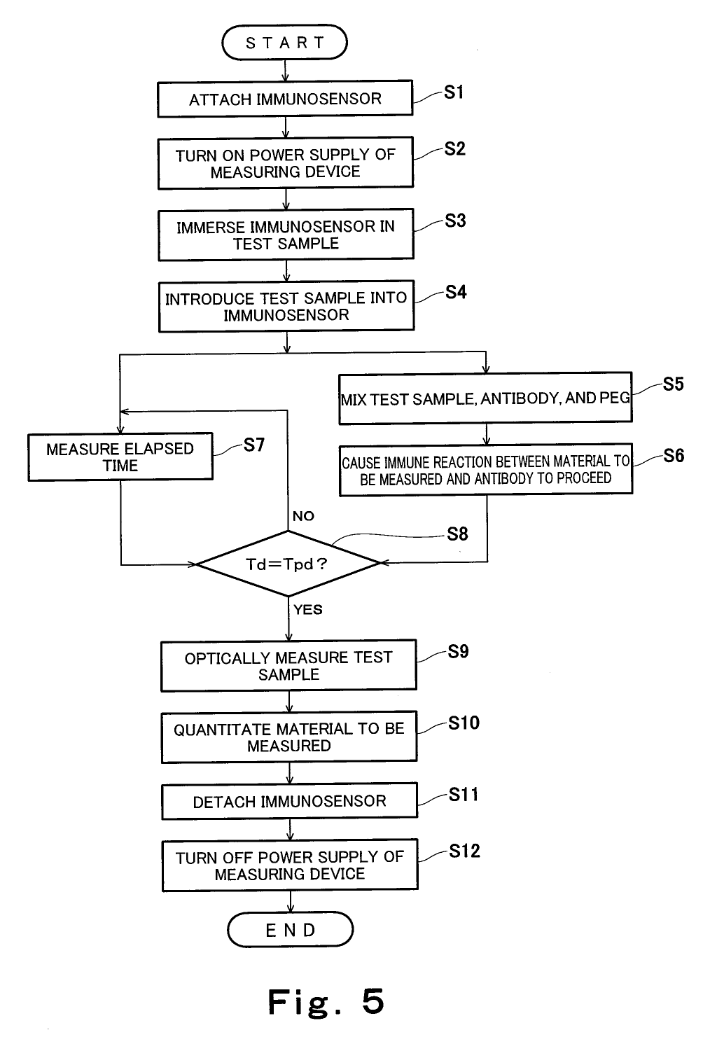

FIG. 5 is a flow chart schematically showing the method for measuring the material to be measured by the measuring device which uses the immunosensor 100 according to Embodiment 1 of the present invention. For convenience sake, FIG. 5 also shows manipulations by the operator associated with the operations of the measuring device, chemical reactions which proceed in accordance with the manipulations by the operator, and the like.

The operator first causes the suction port 104 of the immunosensor 100 to contact the sensor attaching port 305 of the immunosensor attaching portion 301 of the measuring device 300 to attach the immunosensor 100 to the immunosensor attaching portion 301 (Step S1).

When the immunosensor 100 is attached, an immunosensor insertion detecting switch (not shown) that is a micro switch formed inside the immunosensor attaching portion 301 is activated in the measuring device 300. Thus, the controller 401 serving as a control unit detects insertion of the immunosensor 100. With this, a power supply of the measuring device 300 is turned on (Step S2).

Next, for example, the operator immerses the immunosensor 100 in the urine stored in a conveyable container, such as a urine container provided in a toilet bowl or a paper cup, such that at least the sample introducing port 103 is immersed in the urine (Step S3).

Next, the operator presses the sample suction start button 303 of the measuring device 300 to activate the piston mechanism 404. With this, the piston provided inside the piston mechanism 404 moves, so that a predetermined amount (3 mL for example) of the urine is introduced from the sample introducing port 103 of the immunosensor 100 into the sample holding portion 102 (Step S4).

At this time, the urine introduced into the reagent holding portion 102 contacts the anti-albumin antibody of the first reagent body 109 placed closer to the sample introducing port 103, and the anti-albumin antibody is dissolved in the urine. Next, the PEG and the salt, such as sodium hydrogen phthalate, of the second reagent body 110 are dissolved in the urine. As above, since the urine that is the test sample first contacts the anti-albumin antibody contained in the first reagent body 109, the viscosity of the urine does not increase. On this account, the anti-albumin antibody can be easily dissolved in the urine. Moreover, since the second reagent body 110 contains the PEG and the salt, such as sodium hydrogen phthalate, the PEG can be easily dissolved in the urine.

When the first reagent body 109 and the second reagent body 110 are dissolved in the urine, the antigen-antibody reaction between the human albumin that is the antigen contained in the urine and the anti-human albumin antibody proceeds in the sample holding portion 102 of the immunosensor 100 (Step S6).

Meanwhile, when the test sample is introduced into the sample holding portion 102 of the immunosensor 100 in Step S4, the controller 401 of the measuring device 300 causes the timer portion 406 to be activated to start measuring an elapsed time since the start of the introduction of the test sample to the sample holding portion 102 (Step S7).

Next, when the controller 401 of the measuring device 300 determines in accordance with an output signal of the timer portion 406 that an elapsed time Td since the completion of the supply of the test sample to the sample holding portion 102 has reached a predetermined elapsed time Tpd (45 seconds for example) (YES in Step S8), the controller 401 starts an optical measurement of the test sample held in the sample holding portion 102 of the immunosensor 100 (Step S9).

When carrying out the optical measurement of the test sample, the controller 401 of the measuring device 300 causes the light source 407 to irradiate the light incident portion 106 of the immunosensor 100 with light. Specifically, the controller 401 operates such that the light is emitted from the light source 407 through the light incident portion 106 of the immunosensor 100 to the sample holding portion 102, and transmits the urine that is the test sample or scatters by the urine, and the light emanated from the light emanating portion 107 is received by the photoreceiver 408 provided in the measuring device 300 for a predetermined time (50 milliseconds for example).

When the controller 401 of the measuring device 300 determines in accordance with the output signal of the timer portion 406 that the elapsed time Td since the completion of the supply of the test sample to the sample holding portion 102 has not reached the predetermined elapsed time Tpd (NO in Step S8), the controller 401 operates such that the measurement of the elapsed time Td continues.

Then, the controller 401 of the measuring device 300 reads out the calibration curve which is stored in the memory 409 and shows the correlation between the intensity of the emitted light and the concentration of the human albumin, and refers to this calibration curve, thereby converting the intensity of the emitted light received by the photoreceiver 408 into the concentration of the human albumin. With this, the measuring device 300 quantitates the human albumin that is the material to be tested, which is contained in the urine that is the test sample (Step S10).

When the human albumin that is the material to be tested is quantitated in Step S10, the concentration of the human albumin obtained by the above quantitation is displayed on the display portion 302 of the measuring device 300. With this, a user of the measuring device 300 can recognize the completion of the measurement of the concentration of the human albumin contained in the urine. At this time, preferably, the concentration of the human albumin obtained by the above quantitation is stored in the memory 409 together with the time measured by the timer portion 406.

In accordance with the configuration of the measuring device 300 according to the embodiment of the present invention, data regarding the concentration of the human albumin obtained by the quantitation can be recorded in a removable recording medium, such as an SD card, by the recording portion 411. With this, since the user can easily take out the measurement results from the measuring device 300, he or she can bring or send by mail the recording medium to a professional analysis company to request detailed analysis of the measurement results.

Moreover, in accordance with the configuration of the measuring device 300 according to the embodiment of the present invention, the data regarding the concentration of the human albumin obtained by the quantitation can be sent to outside of the measuring device 300 by the sending portion 412. With this, the measurement results can be sent to an analysis related department in a hospital or an analysis related company, and be analyzed by the analysis related department or the analysis related company. Therefore, it is possible to shorten a time elapsing from the measurement to the analysis.

Further, in accordance with the configuration of the measuring device 300 according to the embodiment of the present invention, the measuring device 300 includes the receiving portion 413 which receives an analytical result of the analysis related department or the analysis related company. Therefore, the analytical result can be quickly fed back to the user.

Last, when the operator presses the immunosensor detach button 304 of the measuring device 300, the immunosensor detach mechanism 410 is activated, and the piston inside the piston mechanism 404 moves. With this, the urine held in the sample holding portion 102 of the immunosensor 100a is discharged from the sample introducing port 103 to the toilet bowl or to a base body, such as the paper cup, and the immunosensor 100 is automatically detached from the measuring device 300 (Step S11).

When the immunosensor 100 is detached, the immunosensor insertion detecting switch provided inside the immunosensor attaching portion 301 is activated in the measuring device 300, and the controller 401 detects the detachment of the immunosensor 100. With this, the power supply of the measuring device 300 is turned off (Step S12).

The embodiment of the present invention is configured such that the measuring device 300 causes the test sample to be discharged from the immunosensor 100 and causes immunosensor 100 to be automatically detached therefrom. However, the present embodiment is not limited to this. For example, the embodiment of the present invention may be configured such that the user manually detach the immunosensor 100 from the immunosensor attaching portion 301 without providing a mechanism which causes the immunosensor 100 to be detached and causes the test sample to be discharged.

As above, in accordance with the immunosensor 100 according to Embodiment 1 of the present invention and the measuring device which uses the immunosensor 100, it is possible to suppress the deterioration of the antibody contained in the first reagent body 109 by placing the antibody contained in the first reagent body 109 and the PEG contained in the second reagent body 110 as pure materials in the reagent holding portion 102. Moreover, since the antibody of the first reagent body 109 first contacts the test sample by placing the first reagent body 109 closer to the sample introducing port 103, the antibody can be easily dissolved in the test sample. Further, by containing the salt, such as potassium hydrogen phthalate, in the second reagent body 110, the PEG contained in the second reagent body 110 can be easily dissolved in the test sample. Then, by dissolving the PEG and the metal salt of phthalic acid, such as potassium hydrogen phthalate, in the test sample, it is possible to amplify the response value of the turbidimetric immunoassay and obtain high measurement sensitivity.

Examples of the test sample in the embodiment of the present invention are body fluids, such as serum, blood plasma, urine, interstitial fluid, and lymph fluid, and liquids, such as supernatant liquid of culture medium. Especially, the urine containing urea is preferable as the test sample since daily health control is noninvasively realized at home. Moreover, a mixture of the body fluid and the reagent, such as enzyme or antibody, which reacts with a specific component in the body fluid, or a mixture of the body fluid and a pigment or the like may be introduced into the immunosensor 100 as the test sample.

Moreover, in consideration of a urine qualitative test performed at an initial stage of the health control, a renal function test, a pregnancy test, an ovulation test, and the like, there is a need for measurements of protein, microalbumin, hormones, such as hCG and LH, and the like. For such measurements, the optical measurement based on the antigen-antibody reaction is suitable. Therefore, examples of the material to be measured in the present invention are albumin, hCG, LH, CRP, IgG, and hormones of visceral fat. Moreover, examples of the optical measurement method are methods, such as the nephelometric immunoassay, the turbidimetric immunoassay, and latex agglutination immunoassay, for measuring the degree of turbidity generated in the test sample based on the antigen-antibody reaction.

### EXAMPLES

Hereinafter, Examples of the present invention will be explained while comparing with Comparative Examples in order to facilitate understanding of the effects of the present invention.

### Example 1

In Example 1, the immunosensor 100 according to Embodiment 1 of the present invention was produced in accordance with the above-described producing method.

First, first to fifth antibodies produced from producing cell lines shown in Table 1 were mixed with one another at a weight ratio of 1:1:1:1:1, and the 50 mM potassium hydrogen phthalate aqueous solution (pH 5.0) was adjusted to contain the anti-albumin monoclonal antibody at the concentration of 8 mg/mL. Thus, the anti-albumin antibody reagent was produced. Moreover, the PEG (polyethylene glycol 6000 (produced by Wako Pure Chemical Industries, Ltd.)) having the average molecular weight of 7,300 to 9,300 was added to the 250 mM potassium hydrogen phthalate aqueous solution (pH 5.0) such that the 250 mM potassium hydrogen phthalate aqueous solution was adjusted to contain 20 weight % of the PEG. Thus, the PEG reagent solution was prepared.

TABLE 1

| | Accession Number of Producing |
|---|---|
| | Cell Line Producing Antibody |
| First Antibody | FERM BP-7938 |
| Second Antibody | FERM BP-10460 |
| Third Antibody | FERM BP-10637 |
| Fourth Antibody | FERM BP-10459 |
| Fifth Antibody | FERM BP-7937 |

Next, the base body 101 of the immunosensor 100 was made of transparent polystyrene. At this time, the dimension (inside dimension) of the base body 101 was 8 mm in width, 8 mm in depth, and 45 mm in height.

After the sample supplying port 103 of the base body 101 was closed by the adhesion tape, 125 μL of the anti-albumin antibody reagent solution was poured through the suction port 104 to the lower portion of the reagent holding portion 102, and was frozen in the freezer at −80° C. Thus, the first reagent body 109 was produced. About three hours later, the base body 101 was quickly moved from the freezer to the container which contained the liquid nitrogen, and 100 μL of the PEG reagent solution was poured through the suction port 104 to the reagent holding portion 102. Thus, the second reagent body 110 was formed to contact the upper portion of the first reagent body 109.

Next, the base body 101 in which the first and second reagent bodies 109 and 110 are formed was quickly placed in the chamber of the freeze dryer, and was freeze-dried overnight. Thus, the immunosensor 100 of Example 1 was produced. Last, the suction port 104 of the immunosensor 100 produced as above was sealed with a parafilm (trademark), and the immunosensor 100 was stored at 4° C. in a closed container which contained silica gel.

### Comparative Example 1

The immunosensor 100 was produced in Comparative Example 1 in the same manner as in Example 1 except that the anti-albumin antibody reagent forming the first reagent body 109 and the PEG reagent solution forming the second reagent body were mixed with each other, and the mixture was placed in the reagent holding portion 102. Specifically, 125 μL of the anti-albumin antibody reagent and 100 μL of the PEG reagent solution, which were prepared in Example 1, were mixed with each other to prepare a mixture solution. The mixture solution was poured into the lower portion of the reagent holding portion 102 through the suction port 104, and frozen in the freezer at −80° C. for about three hours. Then, the base body 101 was quickly placed in the chamber of the freeze dryer, and freeze-dried overnight. Thus, the immunosensor 100 of Comparative Example 1 was produced. Last, the suction port 104 of the immunosensor 100 produced as above was sealed with the parafilm (trademark), and the immunosensor 100 was stored at 4° C. in the closed container which contained silica gel.

### Comparative Example 2

The immunosensor 100 was produced in Comparative Example 2 in the same manner as in Example 1 except that the second reagent body 110 was placed below the first reagent body 109 (in other words, the second reagent body 110 was placed closer to the sample introducing port 103 than the first reagent body 109). Specifically, 100 μL of the PEG reagent solution which was prepared in Example 1 was first poured into the lower portion of the reagent holding portion 102 through the suction port 104, and was frozen in the freezer at −80° C. Thus, the second reagent body 110 was produced. Then, 125 μL of the anti-albumin antibody reagent which was prepared in Example 1 was poured through the suction port 104. Thus, the first reagent body 109 was formed to contact the upper portion of the second reagent body 110.

Evaluation Test 1

Measurements of the test samples each containing the human albumin (hereinafter abbreviated as hSA) were carried out using the immunosensor 100 of Example 1 and the immunosensor 100 of Comparative Example 1. Used as the test samples were hSA solutions which contained the hSA at the concentration of 0, 1, 5, 10, 15, and 20 mg/dL, respectively.

First, the immunosensor 100 was taken out from the closed container which contained the silica gel, the parafilm (trademark) which covered the suction port 104 of the immunosensor 100 was removed, and the suction port 104 was connected to a suction pump. Used as the suction pump was a component which carried out suctioning by causing the piston to move by the stepping motor.

Next, after the adhesion tape which closed the sample supplying port 103 was removed, the immunosensor 100 was immersed in the container, which held the test sample, such that the sample supplying port 103 was immersed in the test sample. After the immunosensor 100 was immersed, the suction pump was immediately activated to suction 500 μL of the test sample in 15 seconds into the reagent holding portion 102 through the sample supplying port 103. At this time, a suction rate of the suction pump was about 1,140 μL/sec from the start of the suctioning to about 0.5 second, 10 μL/sec from about 0.5 second to 14.5 seconds, and again 1,140 μL/sec from about 14.5 seconds to 15 seconds.

Then, after 45 seconds from the start of the suctioning of the test sample, the second surface 106 that is the light incident portion 106 was irradiated with 640 nm laser light emitted from the light source 407, and scattered light at 90 degrees emanating from the third surface 107 that is the light emanating portion 107 was measured by the photoreceiver 408.

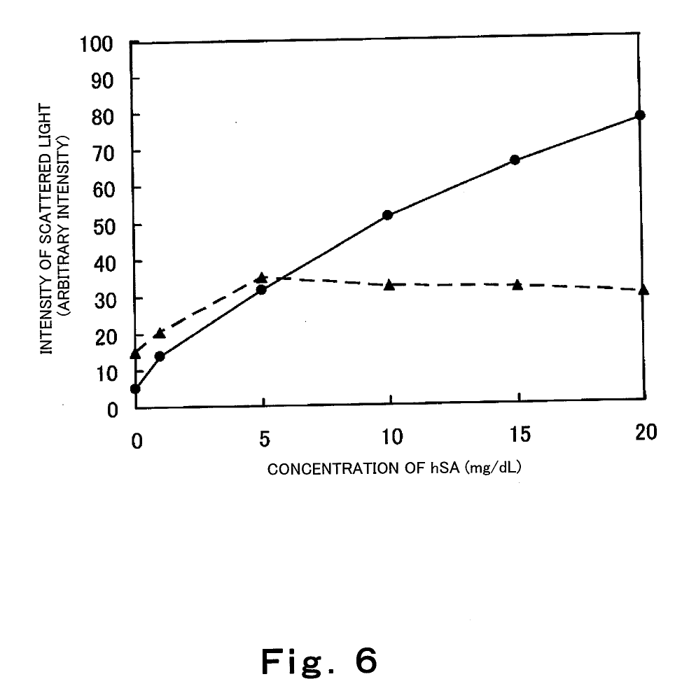

FIG. 6 shows results of Evaluation Test 1 regarding the immunosensor 100 of Example 1 and the immunosensor 100 of Comparative Example 1. In FIG. 6, a horizontal axis shows the concentration (mg/dL) of the hSA in the test sample, a vertical axis shows the intensity (arbitrary intensity) of the scattered light detected by the photoreceiver, data (solid line) shown by black circles shows the result of Example 1, and data (dotted line) shown by black triangles shows the result of Comparative Example 1.

As shown in FIG. 6, in the case of using the immunosensor of Example 1, in the concentration range of 0 to 20 mg/dL, the intensity of the scattered light proportional to the concentration of the antibody could be obtained, and a response characteristic showed satisfactory linearity. In contrast, in the case of using the immunosensor of Comparative Example 1, a blank value defined by the intensity of the scattered light when the test sample whose concentration of the hSA was 0 was higher than that in the case of the immunosensor of Example 1. In addition, the intensity of the scattered light proportional to the concentration of the hSA could not be obtained.

It was confirmed from the above result that the concentration of the albumin in the test sample could be accurately measured by the immunosensor 100 of the present invention.

Evaluation Test 2

How the first and second reagent bodies 109 and 110 placed in the reagent holding portion 102 were dissolved in the test sample was compared between the immunosensor 100 of Example 1 and the immunosensor 100 of Comparative Example 2. Used as the test sample was the aqueous solution whose concentration of the hSA was 0.

A procedure of introducing the test sample into the reagent holding portion 102 herein is the same as that in Evaluation Test 1, so that an explanation thereof is omitted. Note that how the first and second reagent bodies 109 and 110 placed in the reagent holding portion 102 were dissolved in the test sample was visually evaluated after the termination of the suctioning of the test sample.

It was confirmed that in the case of using the immunosensor 100 of Example 1, both the anti-albumin antibody reagent (first reagent body 109) and the PEG reagent (second reagent body 110) which were placed in the reagent holding portion 102 were dissolved in the test sample. In contrast, it was confirmed that in the case of using the immunosensor 100 of Comparative Example 2, about 40% of the anti-albumin antibody reagent remained undissolved.

It was confirmed from the above result that the first and second reagent bodies 109 and 110 could be easily dissolved in the test sample in the immunosensor 100 of the present invention.

### Example 2

The immunosensor 100 was produced in Example 2 in the same manner as in Example 1 by using as the antibody reagents an anti-human albumin monoclonal antibody, an anti-human chorionic gonadotropin monoclonal antibody, and an anti-human C reactive protein monoclonal antibody. Note that these antibodies were produced from the producing cell lines shown in Table 2.

TABLE 2

| | Accession Number of Producing |
|---|---|
| | Cell Line Producing Antibody |
| Anti-human albumin monoclonal | FERM BP-10460 |
| antibody | |
| Anti-human chorionic gonadotropin | FERM BP-6107 |
| monoclonal antibody | |
| Anti-human C reactive protein | FERM BP-6620 |
| antibody | |

### Comparative Example 3

The immunosensor 100 was produced in Comparative Example 3 in the same manner as in Comparative Example 1 by mixing each of the monoclonal antibody reagents used in Example 2 with the PEG reagent.

Evaluation Test 3

Survival rates of respective antibodies were measured by ELISA using the immunosensor 100 of Example 2 and the immunosensor 100 of Comparative Example 3.

Enzyme-Linked Immunosorbent Assay (ELISA)

(A) Coating of Antigens (Human Chorionic Gonadotropin (hCG), Human Albumin (hSA), and Human C Reactive Protein (CRP))

First, a PBS-Az (Az: azide sodium salt) solution containing each of the antigens at the concentration of 0.1 mg/mL was prepared. This solution was poured into a micro plate (polystyrene high-binding flat-bottom plate #2580, produced by Coaster) at 100 μL/well, and was stored overnight in saturated steam at room temperature. Note that the antigen solution was removed by an aspirator immediately before the experiment.

(B) Blocking

A 1 weight % casein-PBS-Az solution was poured at 200 μL/well, and was left for thirty minutes at room temperature. Then, 1 weight % casein-PBS-Az was removed by the aspirator. In the case of not carrying out the following experiment soon, the solution was stored in this state in the saturated steam at 4° C.

(C) Reaction of Antibody

After the parafilm (trademark) covering the suction port 104 of the immunosensor 100 of each of Example 2 and Comparative Example 3 was removed, 100 μL of distilled water was poured through the suction port 104, and the first and second reagent bodies 109 and 110 were dissolved. Next, the solution in which the first and second reagent bodies 109 and 110 were dissolved was diluted by the 1 weight % casein-PBS-Az up to one hundred million times by ten times. Then, each of the diluted solution and the 1 weight % casein-PBS-Az was poured at 50 μL/well into the micro plate coated with the antigens, and the micro plate was left for 120 minutes at room temperature. Then, the micro plate was washed by the PBS, and the remaining PBS was removed by the aspirator three times.

(D) Reaction of Second Antibody

A 1 weight % BSA PBS solution in which 0.2 μg/mL of a peroxidase-labeled goat-derived anti-mouse IgG antibody (produced by KPL) was dissolved or a 1 weight % BSA PBS solution in which 0.2 μg/mL of a peroxidase labeled goat-derived anti-mouse IgM antibody (produced by KPL) was dissolved was poured at 50 μL/well into the micro plate subjected to the antibody reaction, and the micro plate was left for thirty minutes at normal temperature. Then, an operation of washing the micro plate by the PBS and removing the remaining PBS by the aspirator was carried out three times.

(E) Reaction and Stop of Substrate

A solution (substrate solution) obtained by dissolving 40 mg of O-phenylenediamine (for use in biochemical) in 10 mL of a citric acid-phosphoric acid buffer (pH 5) and, immediately before use, adding 4 μL of 30 weight % hydrogen peroxide water was poured at 100 μL/well into the micro plate subjected to the second antibody reaction, and the micro plate was left at room temperature. About three minutes later, 4N sulfuric acid was poured at 25 μL/well to stop the reaction.

(F) Measurement

492 nm light absorbance was measured using a micro plate reader (produced by Toyo Soda).

Note that used as the immunity measuring method in the present Example was Enzyme-Linked Immunosorbent Assay. However, RIA, Fluorescent Antibody Technique, or the like may be used.

(G) Measurement Result

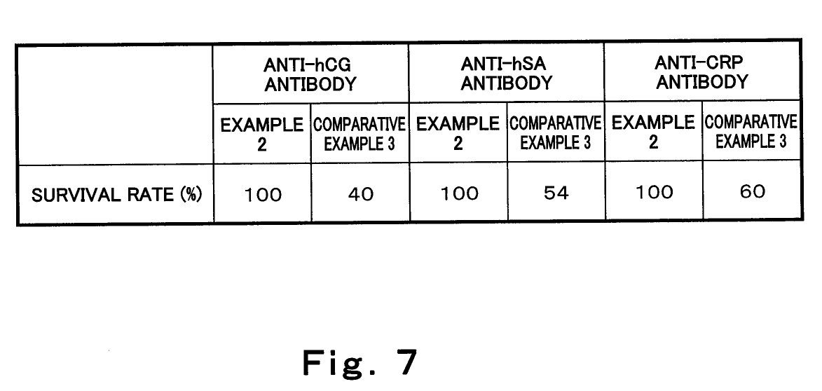

FIG. 7 shows measurement results, which are obtained by ELISA, regarding the immunosensor 100 of Example 2 and the immunosensor 100 of Comparative Example 3. As shown in FIG. 7, in the case of hCQ, the survival rate of the antibody was about 40% in the immunosensor 100 of Comparative Example 3 when it was 100% in the immunosensor 100 of Example 2. In the case of hSA, the survival rate of the antibody was about 54% in the immunosensor 100 of Comparative Example 3 when it was 100% in the immunosensor 100 of Example 2. In the case of CRP, the survival rate of the antibody was about 60% in the immunosensor 100 of Comparative Example 3 when it was 100% in the immunosensor 100 of Example 2. In the case of any antibodies, a preservation performance of the immunosensor 100 of Example 2 was about twice higher than that of the immunosensor 100 of Comparative Example 3.

It was confirmed from the above result that the deterioration of the antibody contained in the first reagent body 109 placed in the reagent holding portion 102 could be suppressed by the immunosensor 100 of the present invention.

### Example 3

The immunosensor 100 was produced in Example 3 in the same manner as in Example 1 except that the PEG reagent solution was prepared as below. Specifically, the PEG reagent solution was prepared such that the weight ratio of the PEG to the potassium hydrogen phthalate was 1:0, 1:0.26, 1:0.38, 1:0.51, 1:0.77, or 1:1.02. The weight ratio of the PEG to the potassium hydrogen phthalate was adjusted by using the PEG aqueous solution and the potassium hydrogen phthalate aqueous solution of pH 5.0, changing the mixing ratio of the PEG aqueous solution and the potassium hydrogen phthalate aqueous solution, and mixing them.

Thus, six types of immunosensors 100 (hereinafter referred to as immunosensors 100A to 100F) were produced using the PEG reagent solutions prepared as above.

Evaluation Test 4

The dissolution rate of the PEG was measured using the immunosensors 100A to 100F of Example 3. Note that water was used as the test sample.

First, as with Evaluation Test 1, the test sample was poured into the reagent holding portion 102. After 45 seconds from the start of the suctioning of the test sample, a portion at which the second reagent body 110 of the reagent holding portion 102 was placed was photographed from the side surface of the base body 101 of the immunosensor. An area of the second reagent body 110, which was not dissolved, in the picture photographed after the suctioning of the test sample was subtracted from an area of the second reagent body 110 in the picture photographed before the suctioning of the test sample. Thus, an area of the second reagent body which was dissolved in the test sample in the reagent holding portion 102 was calculated. Further, a ratio of the area of the second reagent body 110 which was dissolved in the test sample to the area of the second reagent body 110 in the picture photographed before the suctioning of the test sample was calculated as the ratio (dissolution rate) of the dissolved PEG.

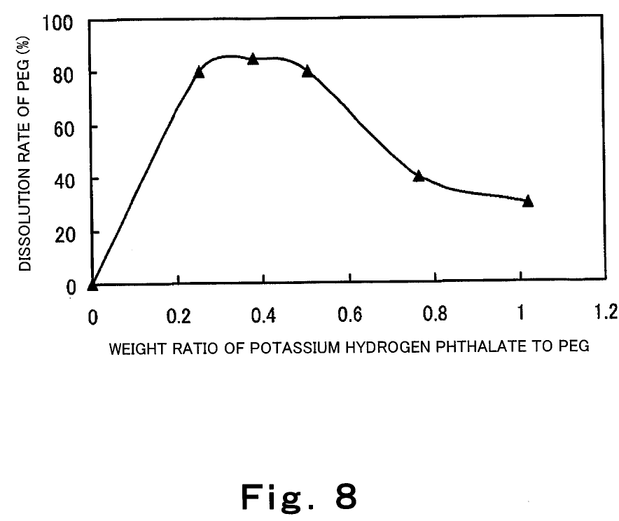

FIG. 8 shows measurement results of the dissolution rate of the PEG in the immunosensor 100 of Example 3. In FIG. 8, a horizontal axis shows the weight ratio of the potassium hydrogen phthalate to the PEG in the PEG reagent (second reagent body 110) of the immunosensor 100, and a vertical axis shows the dissolution rate of the PEG in the second reagent body 110.

As shown in FIG. 8, the dissolution rate of 30% or higher could be obtained in the immunosensors B to F. Especially, in the case of the immunosensors B to D in which the weight ratio of the potassium hydrogen phthalate to the PEG was from 0.26 to 0.51, high dissolution rate of 80% or higher could be obtained. In contrast, in the case of the immunosensor A, the second reagent body 110 containing the PEG reagent was not substantially dissolved in the above procedure, and the dissolution rate was 0.

It was confirmed from the above result that the solubility of the second reagent body 110 was improved by adding the potassium hydrogen phthalate to the second reagent body 110.

Evaluation Test 5

In Evaluation Test 5, the solubility of the PEG which changed depending on the type of the salt contained in the second reagent body 110 together with the PEG was tested.

First, a 40 weight % PEG 6000 aqueous solution and a 500 mM salt solution (potassium hydrogen phthalate, trisodium citrate, disodium succinate, NaCL, or KCL) were mixed with each other at a ratio of 1:1.1 mL of the mixture solution was poured into a 1.5 mL Eppendorf tube (Product Name), and the tube was capped. The mixture solution was frozen in a refrigerator at −80° C. for six hours. Then, the mixture solution was placed in the chamber of the freeze dryer and freeze-dried overnight. Thus, a salt-containing high polymer compound reagent was prepared. After the freeze dry, the tube was immediately closed with a cap, and was stored in the container containing the silica gel until immediately before a solubility test.

The solubility test was carried out as below.

First, an area of the salt-containing high polymer compound reagent was measured by photographing the peripheral surface of the Eppendorf tube (Product Name). Next, the cap of Eppendorf tube (Product Name) was opened, and 1.0 mL of purified water was poured. After thirty seconds to one minute from the completion of the pouring of the purified, pipetting was carried out three times by a micro pipet. Next, the mixture solution was stirred by VoLtex (Product Name) for thirty seconds after one minute thirty seconds from the completion of the pouring of the purified water. After the stirring, the area of the salt-containing high polymer compound reagent which was not dissolved was measured by photographing the peripheral surface of the Eppendorf tube (Product Name). Then, a ratio of the area of the salt-containing high polymer compound reagent which was not dissolved to the area of the salt-containing high polymer compound reagent before the purified water was poured was calculated. Thus, the ratio of the dissolved salt-containing high polymer compound reagent was obtained.

Moreover, as Comparative Example, 1 mL of a 20 weight % PEG 6000 aqueous solution was poured into the 1.5 mL Eppendorf tube (Product Name), the 1.5 mL Eppendorf tube was freeze-dried, and the solubility test was carried out.

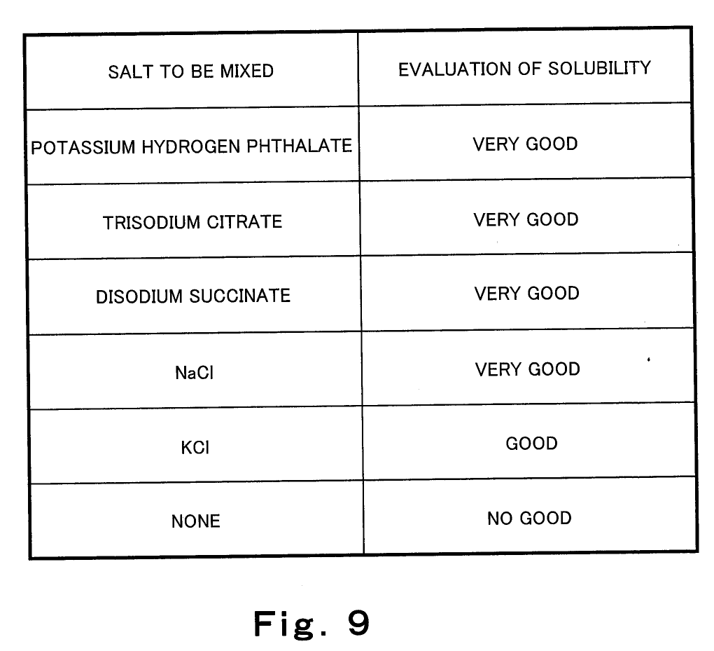

FIG. 9 shows results of the solubility test in Evaluation Test 5. In FIG. 9, “Good” denotes that 80 to 90% of the PEG was dissolved, “Very Good” denotes that 90% or higher of the PEG was dissolved, and “No Good” denotes that the PEG was not dissolved.

It was clear from FIG. 9 that 90% or higher of the PEG was dissolved when the reagent contains potassium hydrogen phthalate, trisodium citrate, disodium succinate, or NaCL together with the PEG, and 80 to 90% of the PEG was dissolved when the reagent contains KCL together with the PEG.

It was confirmed from the above result that the PEG was easily dissolved in the test sample when the second reagent body 110 contains any one of potassium hydrogen phthalate, trisodium citrate, disodium succinate, NaCL, and KCL together with the PEG.

From the foregoing explanation, many modifications and other embodiments of the present invention are obvious to one skilled in the art. Therefore, the foregoing explanation should be interpreted only as an example, and is provided for the purpose of teaching the best mode for carrying out the present invention to one skilled in the art. The structures and/or functional details may be substantially modified within the spirit of the present invention.

### INDUSTRIAL APPLICABILITY

Since the immunosensor according to the present invention and the measuring method using the immunosensor can accurately measure the concentration of the material to be measured, which is contained in the test sample, they are useful in testing fields, especially in medical and medical related testing fields.

1. An immunosensor comprising:
a container-like base body whose internal space forms a sample holding portion which holds a test sample;
a sample introducing port which is formed on the base body to be communicated with the sample holding portion;
a dried first reagent body which contains an antibody to a material to be measured which is contained in the test sample; and
a dried second reagent body which contains polyethylene glycol, wherein
in the sample holding portion, the first reagent body is placed closer to the sample introducing port than the second reagent body.

2. The immunosensor according to claim 1, wherein the first reagent body is placed to be adhered to an inner surface of the base body.

3. The immunosensor according to claim 1, wherein a portion of the second reagent body which is opposed to the first reagent body has a portion which projects toward the second reagent body.

4. The immunosensor according to claim 1, wherein a portion of the second reagent body which is opposed to the first reagent body has a spherical shape.

5. The immunosensor according to claim 1, wherein the second reagent body contains a metal salt of phthalic acid.

6. The immunosensor according to claim 5, wherein the metal salt of phthalic acid is potassium hydrogen phthalate.

7. The immunosensor according to claim 6, wherein a weight ratio of the potassium hydrogen phthalate to the polyethylene glycol is not less than 0.26 and not more than 1.02.

8. The immunosensor according to claim 1, wherein the second reagent body contains a salt selected from the group consisting of potassium hydrogen phthalate, trisodium citrate, disodium succinate, sodium chloride, and potassium chloride.

9. The immunosensor according to claim 1, wherein the base body has a light transmitting portion which transmits light such that the light penetrates a wall forming the base body.

10. A measuring method using an immunosensor comprising: a container-like base body whose internal space forms a sample holding portion which holds a test sample; a sample introducing port which is formed on the base body to be communicated with the sample holding portion; a dried first reagent body which contains an antibody to a material to be measured which is contained in the test sample; and a dried second reagent body which contains polyethylene glycol, wherein in the sample holding portion, the first reagent body is placed closer to the sample introducing port than the second reagent body,
the method comprising the step of introducing the test sample through the sample introducing port to the sample holding portion, wherein: the test sample introduced into the sample holding portion contacts the first reagent body; the first reagent body is dissolved in the test sample; the test sample in which the first reagent body is dissolved contacts the second reagent body; and the second reagent body is dissolved in the test sample.

11. The measuring method using the immunosensor according to claim 10, wherein a concentration of the polyethylene glycol in an entire amount of the test sample introduced into the sample holding portion is not less than 1 weight % and not more than 15 weight %.
